(19) United States
(12) Patent Application Publication
(10) Pub. No.: US 2018/0028510 A1
(43) Pub. Date: Feb. 1, 2018

(54) PREVENTION AND TREATMENT OF THROMBOSIS IN MEDICALLY ILL PATIENTS

(71) Applicant: Portola Pharmaceuticals, Inc., South San Francisco, CA (US)

(72) Inventors: William D. Lis, San Mateo, CA (US); Todd Lorenz, Orinda, CA (US); Uma Sinha, San Francisco, CA (US)

(21) Appl. No.: 15/456,133

(22) Filed: Mar. 10, 2017

Related U.S. Application Data
(63) Continuation of application No. 14/241,824, filed on Apr. 1, 2015, now abandoned.
(60) Provisional application No. 61/529,755, filed on Aug. 31, 2011.

(51) Int. Cl. A61K 31/44

(52) U.S. Cl. CPC A61K 31/44

(57) ABSTRACT

Provided herein are methods for preventing and treating thrombosis in medically ill patients by administering to the patient a therapeutically effective amount of betrixaban.

### CROSS REFERENCE TO RELATED APPLICATIONS

This application is a continuation application of U.S. application Ser. No. 14/241,824, filed Apr. 1, 2015, which is the U.S. national stage application of PCT/US2012/053102, filed Aug. 30, 2012, which claims the benefit under 35 U.S.C. §119(e) of United States Provisional Applications Ser. No. 61/529,755 filed Aug. 31, 2011, the content of which is incorporated by reference in its entirety into the present disclosure.

### FIELD OF THE INVENTION

Provided herein are methods of preventing and treating thrombosis in medically ill patients.

### BACKGROUND

Factor Xa is a serine protease, the activated form of its precursor factor X, and a member of the calcium ion binding, gamma carboxyglutamic acid (GLA)-containing, vitamin K dependent, blood coagulation factors. Factor Xa appears to have a single physiologic substrate, namely prothrombin. Since one molecule of factor Xa may be able to generate greater than 1000 molecules of thrombin (Mann, et al., J. Thrombosis. Haemostasis 1:1504-1514, 2003), direct inhibition of factor Xa as a way of indirectly inhibiting the formation of thrombin has been considered an efficient anticoagulant strategy.

Several classes of small molecule factor Xa inhibitors have been reported, for example, those described in U.S. Pat. Nos. 6,376,515, 7,521,470, and 7,696,352, U.S. Patent Application Publication Nos. 2007/0259924, 2008/0293704, and 2008/0051578, all of which are incorporated by reference in their entirety.

U.S. Pat. Nos. 6,376,515 B2 and 6,835,739 B2, the contents of which are incorporated herein by reference, disclose a specific factor Xa inhibitor compound, betrixaban, having the chemical name of [2-({4-[(dimethylamino)iminomethyl]phenyl}carbonylamino)-5-methoxyphenyl]-N-(5-chloro(2-pyridyl))carboxamide (betrixaban), and chemical structure of:

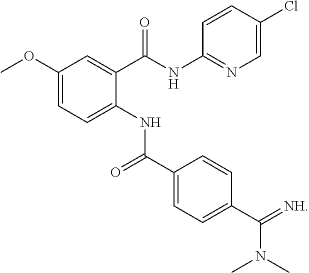

### SUMMARY

The present disclosure provides methods and compositions for the prevention and/or treatment of thrombosis in a medically ill patient. In one embodiments, the method comprises administering to the patient a therapeutically effective amount of betrixaban. In one embodiment, the thrombosis is venous thrombosis.

In one aspect, the patient is at risk of developing a venous thromboembolic disease. In another aspect, the patient suffers from one or more of (a) acutely decompensated heart failure, (b) acute respiratory failure, (c) acute infection without septic shock, (d) an acute rheumatic disorder or (e) cancer. In another aspect, the patient suffers from decreased mobility.

In one aspect, betrixaban is in the form of a pharmaceutically acceptable salt. In a particular aspect, the pharmaceutically acceptable salt of betrixaban is the maleate salt. Betrixaban can be administered in an amount of about 20 mg to about 80 mg per day, or from about 40 mg to about 80 mg per day, and in a particular aspect, 80 mg per day, when administered with food. In another aspect, a 160-mg loading dose is used prior to the daily administration.

### BRIEF DESCRIPTION OF THE DRAWINGS

Provided embodiments are illustrated by way of example, and not limitation, in the figures of the accompanying drawings in which:

FIG. 1 presents comparison of distribution of thrombin inhibition in medically ill patients for apixaban and betrixaban: mean and 5th to 95th percentile; and

FIG. 2 presents comparison of distribution of thrombin inhibition in medically ill patients for rivaroxaban and betrixaban: mean and 5th to 95th percentile.

It will be recognized that some or all of the figures are schematic representations for purposes of illustration. The figures are provided for the purpose of illustrating one or more embodiments with the explicit understanding that they will not be used to limit the scope or the meaning of the claims.

### DETAILED DESCRIPTION

### I. Definitions

Unless defined otherwise, all technical and scientific terms used herein have the same meanings as commonly understood by one of ordinary skill in the art to which this invention belongs. Although any methods and materials similar or equivalent to those described herein can be used in the practice or testing of the present invention, the preferred methods, devices, and materials are now described. All technical and patent publications cited herein are incorporated herein by reference in their entirety. Nothing herein is to be construed as an admission that the invention is not entitled to antedate such disclosure by virtue of prior invention.

As used in the specification and claims, the singular form “a”, “an” and “the” include plural references unless the context clearly dictates otherwise.

As used herein, the term “comprising” is intended to mean that the compositions and methods include the recited elements, but not excluding others. “Consisting essentially of” when used to define compositions and methods, shall mean excluding other elements of any essential significance to the combination. For example, a composition consisting essentially of the elements as defined herein would not exclude other elements that do not materially affect the basic and novel characteristic(s) of the claimed invention. “Consisting of” shall mean excluding more than trace amount of other ingredients and substantial method steps recited. Embodiments defined by each of these transition terms are within the scope of this invention.

The term “treatment” or “treating” means any treatment of a disease or disorder in a subject, such as a mammal, including: preventing or protecting against the disease or disorder, that is, causing the clinical symptoms not to develop; inhibiting the disease or disorder, that is, arresting or suppressing the development of clinical symptoms; and/or relieving the disease or disorder that is, causing the regression of clinical symptoms.

As used herein, the term “preventing” refers to the prophylactic treatment of a patient in need thereof. The prophylactic treatment can be accomplished by providing an appropriate dose of a therapeutic agent to a subject at risk of suffering from an ailment, thereby substantially averting onset of the ailment.

It will be understood by those skilled in the art that in human medicine, it is not always possible to distinguish between “preventing” and “suppressing” since the ultimate inductive event or events may be unknown, latent, or the patient is not ascertained until well after the occurrence of the event or events. Therefore, as used herein the term “prophylaxis” is intended as an element of “treatment” to encompass both “preventing” and “suppressing” as defined herein. The term “protection,” as used herein, is meant to include “prophylaxis.”

The term “therapeutically effective amount” refers to that amount of betrixaban, typically delivered as a pharmaceutical composition, that is sufficient to effect treatment, as defined herein, when administered to a subject in need of such treatment. The therapeutically effective amount will vary depending upon the subject and disease condition being treated, the weight and age of the subject, the severity of the disease condition, the particular compound chosen, the dosing regimen to be followed, timing of administration, the manner of administration and the like, all of which can be determined readily by one of ordinary skill in the art.

As used herein, the term “a medically ill patient” refers to a patient who is admitted to the hospital or a nursing facility for a nonsurgical illness who requires prophylaxis for venous thromboembolic disease, or who is expected to be hospitalized for at least 6 days due to an acute medical condition.

In some embodiments, a medically ill patient meets one or more of the criteria 1-4 and has either at least two venous thromboembolism (VTE) risk factors from a) to q) as outlined below or a D-dimer of more than two times the upper limit of normal.

“Medically ill” criteria may include, but are not limited to:
1. Acutely decompensated heart failure, New York Heart Association (NYHA) class III or IV;
2. Acute respiratory failure without the need for prolonged (<=2 days) respiratory support;
3. Acute infection without septic shock;
4. Acute rheumatic disorders (including acute lumbar pain, sciatica, vertebral compression, acute arthritis of the legs, or an episode of inflammatory bowel disease) or
5. Cancer.

Risk factors for venous thromboembolism (VTE) include, but are not limited to:
a) Age >75 years;
b) Previous history of VTE that required anticoagulant therapy;
c) Expected marked immobilization>=3 days (Level 1—bedrest without bathroom privileges);
d) Obesity (Body Mass Index (BMI)>30 for men or 28.6 for women);
e) Varicose veins or chronic venous insufficiency;
f) Lower extremity paresis;
g) Central venous catheterization;
h) Hormone therapy (antiandrogen, estrogen or selective estrogen receptor modulators (SERMs));
i) Chronic heart failure;
j) Chronic respiratory failure;
k) Active collagen vascular disease;
l) Acute infectious disease contributing to current hospitalization;
m) Erythropoeisis stimulating agents;
n) Inflammatory bowel disease;
o) Venous compression (tumor, hematoma or arterial anomaly);
p) Nephrotic syndrome; and
q) Inherited or acquired thrombophilia.

As used herein, the term “thrombosis” refers to the formation of a blood clot inside a blood vessel, obstructing the flow of blood through the circulatory system. In some aspects, the thrombosis is “venous thrombosis” which is a blood clot that forms within a vein.

As used herein, the term “patient” or “subject” refers to mammals and includes humans and non-human mammals. In particular embodiments herein, the patient or subject is a human.

The term “about” when used before a numerical value indicates that the value may vary within reasonable range, such as ±5%, ±1%, and ±0.2%.

### II. Methods

It is contemplated and has been confirmed with modeling, as described in Examples 1 and 2 that betrixaban is useful in preventing and treating thrombosis in medically ill patients. It is further contemplated that no safety concern would be raised in these patients even though such patients are at impaired physiological conditions. It is also observed that, unlike other fXa inhibitors, betrixaban can be administered to medically ill patients at the same dose as for normal patients, when it is administered with food.

As described above, a medically ill patient is one that is at risk of developing a venous thromboembolic disease. In another aspect, the patient suffers from one or more of (a) acutely decompensated heart failure, (b) acute respiratory failure, (c) acute infection without septic shock, (d) an acute rheumatic disorder or (e) cancer. In another aspect, the patient suffers from decreased mobility.

In some embodiments, the acutely decompensated heart failure is New York Heart Association (NYHA) class III or IV. In some embodiments, the acute respiratory failure is without the need for prolonged (<=2 days) respiratory support. In some embodiments, the acute infection is without septic shock. In some embodiments, acute rheumatic disorders (including acute lumbar pain, sciatica, vertebral compression, acute arthritis of the legs, or an episode of inflammatory bowel disease).

Thus, one embodiment of the disclosure provides a method for the prevention or treatment of thrombosis in a medically ill patient, comprising administering to the patient a therapeutically effective amount of betrixaban.

In one embodiment, the patient is at risk of developing a venous thromboembolic disease. In another embodiment, the patient suffers from decreased mobility. In another embodiment, the thrombosis is venous thrombosis.

In some aspects, thrombosis is a feature of an underlying disease or condition. Non-limiting examples of such disease or condition include acute coronary syndrome, myocardial infarction, unstable angina, refractory angina, occlusive coronary thrombus occurring post-thrombolytic therapy or post-coronary angioplasty, a thrombotically mediated cerebrovascular syndrome, embolic stroke, thrombotic stroke, thromboembolic stroke, systemic embolism, ischemic stroke, venous thromboembolism, atrial fibrillation, non-valvular atrial fibrillation, atrial flutter, transient ischemic attacks, venous thrombosis, deep venous thrombosis, pulmonary embolus, coagulopathy, disseminated intravascular coagulation, thrombotic thrombocytopenic purpura, thromboanglitis obliterans, thrombotic disease associated with heparin-induced thrombocytopenia, thrombotic complications associated with extracorporeal circulation, thrombotic complications associated with instrumentation, thrombotic complications associated with the fitting of prosthetic devices, occlusive coronary thrombus formation resulting from either thrombolytic therapy or percutaneous transluminal coronary angioplasty, thrombus formation in the venous vasculature, disseminated intravascular coagulopathy, a condition wherein there is rapid consumption of coagulation factors and systemic coagulation which results in the formation of life-threatening thrombi occurring throughout the microvasculature leading to widespread organ failure, hemorrhagic stroke, renal dialysis, blood oxygenation, and cardiac catheterization.

In some embodiments, the disease or condition is selected from the group consisting of embolic stroke, thrombotic stroke, venous thrombosis, deep venous thrombosis, acute coronary syndrome, and myocardial infarction.

Betrixaban can be administered in an amount of about 20 mg to about 80 mg per day, or about 40 mg to about 80 mg per day. In one aspect, betrixaban is administered when the patient is in a fasted state. In a particular aspect, betrixaban is administered 80 mg per day, with food. In another aspect, a 160-mg loading dose is used prior to the daily administration.

In some embodiments, a P-glycoprotein (Pgp) inhibitor is concomitantly administered to the patient. It is discovered that Pgp inhibitor increases the exposure of betrixaban, and therefore co-administration with a Pgp inhibitor enables betrixaban to be administered at a subtherapeutic dose, or alternatively a synergistically effective dose. In one aspect, when used together with a Pgp inhibitor, betrixaban is administered at about 40 mg, or alternatively about 35 mg, or 30, or 25, or 20, or 15, or 10 mg daily. The same amount is applicable to patients who experience kidney insufficiency.

Examples of Pgp inhibitors include but are not limited to amiodarone, ketoconazole, clarithromycin, verapamil, diltiazem, cyclosporine, quinidine, erythromycin, itraconazole, ivermectin, mefloquine, nifedipine, ofloxacin, propafenone, ritonavir, tacrolimusvalspodar (PSC-833), zosuquidar (LY-335979), elacridar (GF120918), HM30181AK, R101933, and R102207, or a pharmaceutically acceptable salt thereof.

Besides being useful for human treatment, betrixaban is also contemplated to be useful for veterinary treatment of companion animals, exotic animals and farm animals, including mammals, rodents, and the like. More preferred animals include horses, dogs, and cats.

### III. Betrixaban, its Salts and Crystalline Polymorph Form

Betrixaban has the chemical name of [2-({4-[dimethylamino)iminomethyl]phenyl}carbonylamino)-5-methoxyphenyl]-N-(5-chloro(2-pyridyl))carboxamide and has been disclosed as Example 206 in U.S. Pat. Nos. 6,376,515 and 6,835,739, both of which are incorporated by reference in their entirety herein. Further descriptions of salts and polymorphs of salts of betrixaban can be found in U.S. Pat. No. 7,598,276, which is incorporated by reference in its entirety herein.

In a specific embodiment, the salt of betrixaban is a maleate salt. The maleate salt be formed by protonating one or more nitrogen atoms of betrixaban. In one embodiment, the amidino nitrogen (=NH) of betrixaban is protonated (=NH₂⁺) to form the salt. In some embodiments, the aggregate daily dose of the factor Xa inhibitor is 80 mg of betrixaban and in some embodiments, the 80 mg of betrixaban is administered in the form of a salt, for example the maleate salt.

In one embodiment, the maleate salt of betrixaban is represented by Formula I:

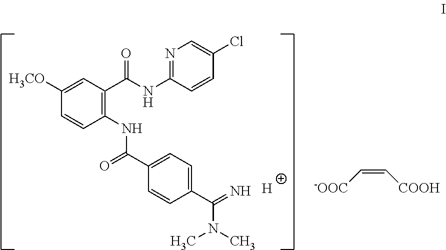

This is also referred to herein as betrixaban maleate. In one embodiment, the aggregate daily dose is about 20 to about 80 mg of betrixaban maleate. In another embodiment, the aggregate daily dose is about 40 mg, 60 mg, or 80 mg aggregate of betrixaban maleate. In another embodiment, betrixaban is administered with food; that is, betrixaban is administered together with food, or within a short period before or after food consumption. A “short period” as used here, refers to an amount of time that is about 30 minutes or shorter, or alternatively about 25, 20, 15, 20, or 5 minutes or shorter.

In another embodiment, the salt of betrixaban has a crystalline polymorph form (Form I). Form I exhibits a powder X-ray diffraction pattern having at least four and more preferably eight of the following approximate characteristic peak locations: 4.9, 9.7, 13.8, 14.1, 15.2, 17.6, 18.5, 20.8, 21.6, 22.7, 24.1, 26.3, 26.8 degrees 2θ. In still another embodiment, the powder X-ray diffraction pattern has approximate characteristic peak locations of 4.9, 9.7, 11.8, 13.8, 14.1, 15.2, 17.6, 18.5, 19.9, 20.8, 21.6, 22.7, 24.1, 25.0, 26.3, 26.8 degrees 2θ. Form I is further described in U.S. Pat. No. 7,598,276, which is incorporated by reference in its entirety herein. In some embodiments, Form I has a melting point of 201° C.

In another embodiment, the salt of betrixaban has a crystalline polymorph form, Form II. In some embodiments, Form II is an anhydrate. In some embodiments, Form II is characterized by properties including one or more of the following as described in details herein:
its X-ray powder diffraction pattern (XRPD);
its infrared spectrum (IR);
its differential scanning calorimetry (DSC);
its thermogravimetric analysis (TGA);
its vapor sorption curve,
solid state NMR, and
crystal structure, such as unit cell structure.

In one embodiment, Form II exhibits an X-ray powder diffraction pattern having the following approximate characteristic peak locations: 5.0, 9.7, 10.1, 15.3, 17.5, and 19.6 degrees 2θ. In another embodiment, the X-ray powder diffraction pattern has at least four, six, eight or ten of the approximate characteristic peak locations of 5.0, 9.7, 10.1, 14.6, 15.3, 17.5, 18.0, 18.7, 19.2, 19.6, 22.0, 22.6, 23.0, 23.7, 24.5, 26.5, 26.9, 29.2, 29.5, 30.4 and 35.0 degrees 2θ. In another embodiment, the X-ray powder diffraction pattern has at least four, six, eight or ten of the approximate characteristic peak locations of 5.0, 9.5, 9.7, 10.1, 14.6, 15.3, 17.5, 18.0, 18.7, 19.2, 19.6, 22.0, 22.6, 23.0, 23.7, 24.5, 26.5, 26.9, 29.2, 29.5, 30.4 and 35.0 degrees 2θ. In another embodiment, the X-ray powder diffraction pattern has at least four, six, eight or ten of the approximate characteristic peak locations of 15.3, 5.0, 10.1, 17.5, 9.7, 19.6, 24.5, 18.6, 18.0, 14.5, 22.6, 22.9, 23.0, 22.1, 29.2, 26.5, 24.8, 18.3, and 21.6 degrees 2θ. It is contemplated that the approximate characteristic peaks will have a deviation of up to about 0.1 or 0.05 degrees 2θ.

In one embodiment, Form II is an anhydrous crystalline form. In some embodiments, it is a white solid with high melting point (213° C.). It is the most thermodynamically stable form known to date and is monotropically related to the polymorph Form I disclosed in the '276 Patent. It absorbs up to 1% water at 95% RH.

In another aspect, there is provided a crystalline polymorph Form III of the maleate salt of betrixaban. In some embodiments, Form III is characterized by properties including one or more of the following as described in details herein:
its X-ray powder diffraction pattern (XRPD);
its infrared spectrum (IR);
its differential scanning calorimetry (DSC);
its thermogravimetric analysis (TGA);
its vapor sorption curve;
solid state NMR, and
crystal structure, such as unit cell structure.

In some embodiments, Form III exhibits an X-ray powder diffraction pattern having at least the following approximate characteristic peak locations 15.1, 2.2, 4.9, 17.4, 10.0, and 22.4 degrees 2θ. In one embodiment, the X-ray powder diffraction pattern is characterized with peaks having a relative intensity of 10% or more: 15.1, 2.2, 4.9, 17.4, 10.0, 22.4, 26.5, and 2.9 degrees 2θ. In another embodiment, the X-ray powder diffraction pattern has at least six, or eight, or ten, or all of the approximate characteristic peak locations selected from 15.1, 2.2, 4.9, 17.4, 10.0, 22.4, 26.5, 2.9, 24.6, 19.4, 24.2, 16.3, 20.7, 22.9, 29.0, 9.6, 18.0, 18.5, 29.3, 22.0, and 30.3 degrees 2θ. In another embodiment, the X-ray powder diffraction pattern has at least four, six, eight, ten or all of the approximate characteristic peak locations of 15.1, 2.2, 4.9, 17.4, 10.0, 22.4, 26.5, 2.9, 24.6, 19.4, 24.2, 16.3, 20.7, 22.9, 29.0, 9.6, 18.0, 18.5, and 29.3 degrees 2θ.

In some embodiments, Form III is a hydrate. In some embodiments, Form III is a hemihydrate. In some embodiments, the Form III is a channel hydrate.

In some embodiments, Form III is a hemihydrate with two independent salt pairs of betrixaban and maleic acid in a crystallographic asymmetric unit. The two cations have dissimilar overall conformations arising from a substantial rotation about the N1—C11 bond of approximately 100 degrees. In some embodiments, the crystal structure is characterized by a unit cell structure with the following cell parameters at 100 K:

| | | |
|---|---|---|
| a = 8.2369(4) Å | α = 107.045(4) ° | V = 2675.7(2) Å³ |
| b = 18.3639(9) | β = 93.758(4) | Space group = P1, #2 |
| c = 18.5623(9) | γ = 91.459(4) | Z = 2 |

Betrixaban can be prepared according to methods described in U.S. Pat. Nos. 6,376,515 and 7,598,276, and U.S. Patent Application No. 12/969,371, filed Dec. 15, 2010, all of which are hereby incorporated by reference in their entirety. Preparation of the maleate salt of betrixaban and Form I is described in U.S. Pat. No. 7,598,276. Preparation of Forms II and III is described in U.S. Patent Application Publication No. 2012/0071519.

### IV. Formulations

Another aspect of the invention provides a unit dose formulation comprising betrixaban in an amount of about 80 mg of betrixaban maleate, or alternatively about 40 mg, or 20 mg of betrixaban maleate. In some embodiments, the aggregate daily dose is formulated for administration to the patient once or twice daily. In some embodiments, the unit dose formulation further comprises a pharmaceutically acceptable carrier. In some embodiments, the formulation further includes a P-glycoprotein inhibitor. In one such aspect, the amount of betrixaban in the formulation is a synergistically effective amount.

The compositions of this invention may be in the form of tablets, capsules, lozenges, or elixirs for oral administration, suppositories, sterile solutions or suspensions or injectable administration, and the like, or incorporated into shaped articles. The method of administration will vary from subject to subject and be dependent upon such factors as the type of mammal being treated, its sex, weight, diet, concurrent medication, overall clinical condition, the particular compounds and/or salts employed, the specific use for which these compounds and/or salts are employed, and other factors which those skilled in the medical arts will recognize.

Capsules useful in the present invention can be prepared using conventional and known encapsulation techniques, such as that described in Stroud et al., U.S. Pat. No. 5,735,105. The capsule is typically a hollow shell of generally cylindrical shape having a diameter and length sufficient so that the pharmaceutical solution compositions containing the appropriate dose of the active agents fit inside the capsule. The exterior of the capsules can include plasticizer, water, gelatin, modified starches, gums, carrageenans, and mixtures thereof. Those skilled in the art will appreciate what compositions are suitable.

In addition to the active agents, tablets useful in the present invention can comprise fillers, binders, compression agents, lubricants, disintegrants, colorants, water, talc and other elements recognized by one of skill in the art. The tablets can be homogeneous with a single layer at the core, or have multiple layers in order to realize preferred release profiles. In some instances, the tablets of the instant invention may be coated, such as with an enteric coating. One of skill in the art will appreciate that other excipients are useful in the tablets of the present invention.

Formulations suitable for delivery through a nasogastric tube are also contemplated. Administration using a nasogastric tube is useful considering that the medically ill patient may not be able to receive betrixaban orally. In some aspects, betrixaban is mixed with nutritionally ingredients that the medically ill patient takes as food supplement.

Lozenges useful in the present invention include an appropriate amount of the active agents as well as any fillers, binders, disintegrants, solvents, solubilizing agents, sweeteners, coloring agents and any other ingredients that one of skill in the art would appreciate is necessary. Lozenges of the present invention are designed to dissolve and release the active agents on contact with the mouth of the patient. One of skill in the art will appreciate that other delivery methods are useful in the present invention.

Formulations of this invention are prepared for storage or administration by mixing active agents having a desired degree of purity with physiologically acceptable carriers, excipients, stabilizers etc., and may be provided in sustained release or timed release formulations. Acceptable carriers or diluents for therapeutic use are well known in the pharmaceutical field, and are described, for example, in Remington's Pharmaceutical Sciences, Mack Publishing Co., (A. R. Gennaro Ed. 1985). Such materials are nontoxic to the recipients at the dosages and concentrations employed, and include buffers such as phosphate, citrate, acetate and other organic acid compounds and/or salts, antioxidants such as ascorbic acid, low molecular weight (less than about ten residues) peptides such as polyarginine, proteins, such as serum albumin, gelatin, or immunoglobulins, hydrophilic polymers such as polyvinylpyrrolidinone, amino acids such as glycine, glutamic acid, aspartic acid, or arginine, monosaccharides, disaccharides, and other carbohydrates including cellulose or its derivatives, glucose, mannose or dextrins, chelating agents such as EDTA, sugar alcohols such as mannitol or sorbitol, counterions such as sodium, and/or nonionic surfactants such as Tween, Pluronics or polyethyleneglycol.

Preferably, dosage formulations of the invention to be used for therapeutic administration are sterile. Sterility is readily accomplished by filtration through sterile membranes such as 0.2 micron membranes, or by other conventional methods. Formulations typically will be stored in lyophilized form or as an aqueous solution. The pH of the preparations of this invention typically will be between 3 and 11, more preferably from 5 to 9 and most preferably from 7 to 8. It will be understood that use of certain of the foregoing excipients, carriers, or stabilizers may result in the formation of cyclic polypeptide compounds and/or salts. Route of administration may be by injection, such as intravenously (bolus and/or infusion), subcutaneously, intramuscularly, or colonically, rectally, nasally or intraperitoneally. Other dosage forms such as suppositories, implanted pellets or small cylinders, aerosols, oral dosage formulations (such as tablets, capsules and lozenges) and topical formulations such as ointments, drops and dermal patches may be used. The sterile membranes may be desirably incorporated into shaped articles such as implants which may employ inert materials such as biodegradable polymers or synthetic silicones, for example, Silastic, silicone rubber or other polymers commercially available.

The compositions of this invention may be in the form of liposome delivery systems, such as small unilamellar vesicles, large unilamellar vesicles and multilamellar vesicles. Liposomes can be formed from a variety of lipids, such as cholesterol, stearylamine or phosphatidylcholines.

The compositions of this invention may also be delivered by the use of antibodies, antibody fragments, growth factors, hormones, or other targeting moieties, to which the salt molecules are coupled. The compositions of this invention may also be coupled with suitable polymers as targetable drug carriers. Such polymers can include polyvinylpyrrolidinone, pyran copolymer, polyhydroxy-propyl-methacrylamide-phenol, polyhydroxyethyl-aspartamide-phenol, or polyethyleneoxide-polylysine substituted with palmitoyl residues. Furthermore, compositions of the invention may be coupled to a class of biodegradable polymers useful in achieving controlled release of a drug, for example polylactic acid, polyglycolic acid, copolymers of polylactic and polyglycolic acid, polyepsilon caprolactone, polyhydroxy butyric acid, polyorthoesters, polyacetals, polydihydropyrans, polycyanoacrylates and cross linked or amphipathic block copolymers of hydrogels. Polymers and semipermeable polymer matrices may be formed into shaped articles, such as valves, stents, tubing, prostheses and the like.

In some embodiments, an amiodarone tablet comprises amiodarone hydrochloride, lactose monohydrate, magnesime stearate, povidone, pregelatinized corn starch, sodium starch glycolate, steric acid, and opotionally one or more coloring agents.

### V. Dosing

Therapeutically effective dosages may be determined by either in vitro or in vivo methods. The optimal dosage required may be determined according to the patient's condition, age, gender, weight, etc. The range of therapeutically effective dosages will be influenced by the route of administration, the therapeutic objectives and the condition of the patient. Accordingly, it may be necessary for the therapist to titer the dosage and modify the route of administration as required to obtain the optimal therapeutic effect. The determination of effective dosage levels, that is, the dosage levels necessary to achieve the desired result, will be readily determined by one skilled in the art. Typically, applications of betrixaban are commenced at lower dosage levels, with dosage levels being increased until the desired effect is achieved.

Typically, about 0.5 to 500 mg of betrixaban provided herein is combined with a physiologically acceptable vehicle, carrier, excipient, binder, preservative, stabilizer, dye, flavor etc., as called for by accepted pharmaceutical practice. The amount of active ingredient in these compositions is such that a suitable dosage in the range indicated is obtained.

The formulations provided herein may be administered once or several times daily and other dosage regimens may also be useful. U.S. Patent Application Publication No. 2008/0153876 provides detailed betrixaban dosing information, which is hereby incorporated by reference in its entirety.

In some embodiments, the dosage is an aggregate daily dose of between 20 mg and 80 mg of betrixaban or betrixaban maleate salt and may be administered once, twice or three times daily. In some embodiments, the dosage is an aggregate daily dose of between 20 mg and 80 mg and may be administered once, twice or three times daily. In some embodiments, the dosage is an aggregate daily dose of 20, 30, 40, 50, 60, 70, or 80 mg and may be administered once, twice or three times daily, preferably once or twice daily. In some embodiments, the dosage is an aggregate daily dose of 20, 40 or 80 mg and may be administered once or twice daily, preferably once daily.

### EXAMPLES

The present technology is further defined by reference to the following examples. It will be apparent to those skilled in the art that many modifications, both to compositions and methods, may be practiced without departing from the scope of the current invention.

### Example 1

This example proposes a clinical trial to test betrixaban's ability to prevent and treat thrombosis in medically ill patients.

This is a multi-center, randomized trial with double-blind, double-dummy betrixaban ×90 days, vs. Enoxaparin ×10 days short and long term assessment. The recruitment duration is 24 months enrollment, 90 days treatment and followed by 7-10 days follow up (total study approx 28 months). Treatment schedule is as follows: first 10 days enoxaparin vs. betrixaban; then betrixaban vs. placebo in addition to standard therapy till 90 days.

This trial recruits 6.850 patients with 3,425 in each arm. The patients are recruited on 475 sites (enrollment rate=0.6; Benchmark—Rate 0.6—Adjusted Magellan) in the United States, Canada and Europe (8-10 countries in Europe).

In each arm, patients are randomly selected for receiving betrixaban QD or Enoxaparin SubQ in addition to standard therapy or to betrixaban and placebo after discharge on day 10 (Note: 2 lead-in doses to start).

There are six visits per patient: Day 10, Day 35, Day 60, Day 90 and Follow up/EOS. The visits include physical examination, medical history, ECG, complete blood chemistry as well as hematocrit, hemoglobin, liver function tests (LFTs). Pharmacokinetics sample are collected at Day 10 and 35. D-dimer samples are collected at Screen, Day 10, 35, 60 and 90.

Study intervention includes Betrixaban vs. matched placebo (and blinded lovenox/betrixaban during the initial 10 days and any subsequent hospitalizations during the 90-day treatment period).

Patients selected for this trial are subjects 40 yrs or older and at risk for thrombosis due to hospitalization and/or decreased level of mobility due to one or more the following underlying conditions:
acute infectious diseases;
heart failure;
cancer; or
acute ischemic stroke.

Exclusion criteria include:
Known atrial fibrillation (AF);
Life expectancy<1 year at entry;
Preplanned surgery within less than 90 days from entry; or
Recent hemorrhagic events.

The primary endpoint of the trial includes composite of asymptomatic Proximal Deep Vein Thrombosis (DVT), Symptomatic deep vein thrombosis DVT, Symptomatic non fatal PE and VTE related death for NI at 10 days and for superiority at 35 days (in a closed test procedure).

The secondary endpoint includes re-hospitalization, all cause mortality, VTE related death, bleeding rates after 10 days, after 35 days, after 60 days, after 90 days and at study end between 10-90 days and 90 to study end.

This clinical trial will provide data to demonstrate that betrixaban is efficacious in the prevention thromboembolic events in medically ill patients. Such efficacy is further proven to be superior to the standard of car. Further, once a patient develops thrombosis, the data will show that betrixaban is effective in treating the thrombosis.

This example will also establish the efficacy of betrixaban for the prevention of re-hospitalization vs. standard of care in the medically ill population and establish the safety (major and clinically relevant bleeding) of betrixaban for the prevention of thromboembolic events in the medically ill population.

### Example 2

This example uses modeling to show that the appropriate dosing of betrixaban for medically ill patients can be 80 mg QD with food.

Clinical studies have been performed with rivaroxaban in acute medically ill patients (MAGELLAN), and a similar trial is concluding in patients that have been treated with apixaban (ADOPT). Within the clinical development programs of both apixaban and rivaroxaban, studies of venous thromboembolism (VTE) prophylaxis in both the post-orthopedic surgery and the acute medically ill populations have used doses that were half the doses used in studies of patients with atrial fibrillation. Specifically, the ROCKET study of rivaroxaban for atrial fibrillation used 20 mg QD, as compared to 10 mg QD in the MAGELLAN study in acute medically ill patients. The ARISTOTLE study of apixaban for atrial fibrillation used 5 mg BID, while 2.5 mg BID was used in the ADVANCE 2 and 3, studies in patients post-orthopedic surgery and the ongoing ADOPT study in patients with acute medical illness.

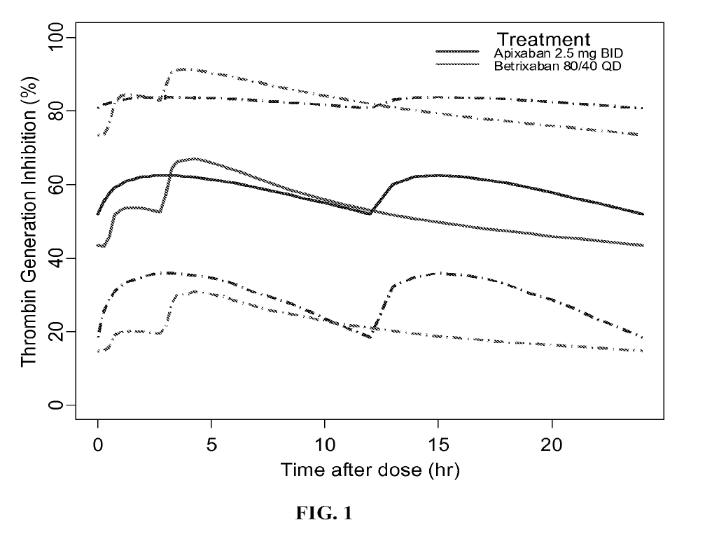

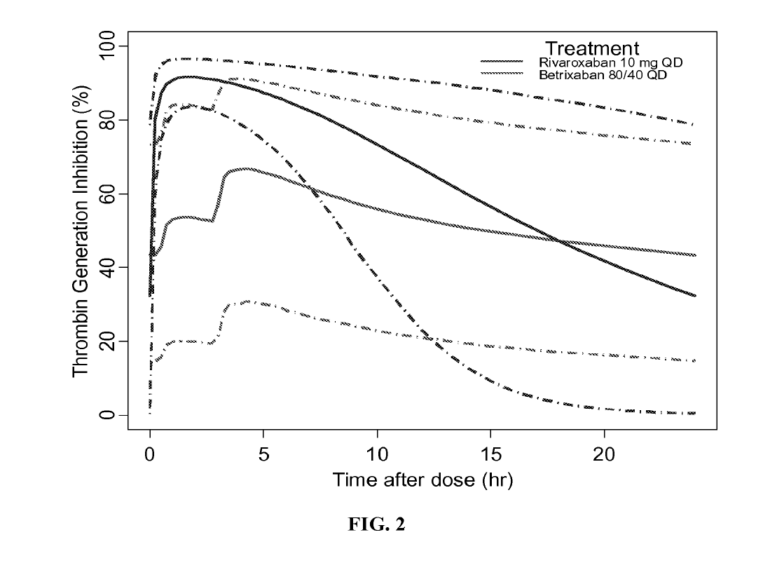

This example used computer modeling which identified an 80 mg dose of betrixaban given once daily with food as providing a pharmacodynamic profile very similar to the 2.5 mg BID dose of apixaban used in the ADOPT study. FIG. 1-2 show the predicted level of thrombin generation inhibition over time for betrixaban 80 mg (40 mg in patients on Pgp inhibitors or renal insufficiency) QD and apixaban 2.5 mg BID (FIG. 1) or rivaroxaban 10 mg QD (FIG. 2).

It is contemplated that the 80 mg daily dose follows a 160 mg loading dose. It is also noted that the 80 mg daily dose is administered with food.

Although the foregoing has been described in some detail by way of illustration and example for purposes of clarity of understanding, one of skill in the art will appreciate that certain changes and modifications may be practiced within the scope of the appended claims. In addition, each reference provided herein is incorporated by reference in its entirety to the same extent as if each reference was individually incorporated by reference.

1. A method for the prevention or treatment of thrombosis in a medically ill patient, comprising administering to the patient a therapeutically effective amount of betrixaban.

2. The method of claim 1, wherein the patient is at risk of developing a venous thromboembolic disease.

3. The method of claim 1, wherein the patient suffers from one or more of (a) acutely decompensated heart failure, (b) acute respiratory failure, (c) acute infection without septic shock, (d) an acute rheumatic disorder or (e) cancer.

4. The method of claim 1, wherein the patient suffers from decreased mobility.

5. The method of claim 1, wherein betrixaban is administered to the patient once daily or twice daily.

6. The method of claim 1, wherein betrixaban is in the form of a pharmaceutically acceptable salt.

7. The method of claim 6, wherein the pharmaceutically acceptable salt of betrixaban is a maleate salt.

8. The method of claim 1, wherein betrixaban is administered in an amount of about 20 mg to about 80 mg per day.

9. The method of claim 1, wherein betrixaban is administered in an amount of about 40 mg to about 80 mg per day.

10. The method of claim 1, wherein betrixaban is administered with food.

11. The method of claim 1, wherein the patient receives about 160 mg of betrixaban prior to the administration.

12. The method of claim 1, wherein the thrombosis is venous thrombosis.
